(19) United States
(12) Patent Application Publication
(10) Pub. No.: US 2006/0292031 A1
(43) Pub. Date: Dec. 28, 2006

(54) Method of inactivating viruses associated with biomaterial

(76) Inventors: Yung-Ho Chiu, Taipei County (TW); Kuo-Ching Lin, Taipei County (TW); Yu-Ting Chen, Taipei County (TW); Zui-Yeh Lin, Taipei County (TW); Chi-Min Chen, Taipei County (TW); Meng-Yang Chang, Taipei County (TW)

Correspondence Address: MORRISON & FOERSTER LLP, 755 PAGE MILL RD, PALO ALTO, CA, 94304-1018, US

(21) Appl. No.: 11/180,278

(22) Filed: Jul. 12, 2005

(30) Foreign Application Priority Data
Jul. 12, 2004 (TW) 093120762
Mar. 16, 2005 (TW) 094107971

(51) Int. Cl. A61L 2/18

(52) U.S. Cl. 422/33; 422/37; 422/36

(57) ABSTRACT

The present invention provides a safe, efficient, nonpolluting and economical method for inactivating viruses in a biomaterial, characterized in comprising a step of introducing a high-pressure fluid into a container containing a biomaterial to be treated at a speed capable of forming non-turbulent flow with Reynold's number of 2000 or less, thereby inactivating viruses potentially existing in the biomaterial. The method in accordance with the present invention can inactivate or eliminate viruses in a heat-sensitive biomaterial such as proteinous material, without decreasing the biological activities of the biomaterial significantly.

### FIELD OF THE INVENTION

The present invention relates to methods for inactivating of viruses, and more particularly, to a disinfection method against the virus within a biomaterial.

### BACKGROUND OF THE INVENTION

To disinfect virus contaminate within a biomaterial is a critical step for producing medicinal products with quality. However, to inactivate a biological “live” substance, a virus particle, from or within a raw material without interfering another biological active substance is difficult, as most of the known disinfective procedures require extreme conditions such as a high temperature, a high or a low pH, or a disinfectant to kill target microorganisms. As a result, microorganisms are not the only target to be destroyed but also all ingredients thereof are the targets to be destroyed. To create a method that to kill target microorganism without or only minor interfere the main function of the bio-ingredient is essential in the development of the medicinal product in the future.

Therefore, a virus disinfection method that is safe, efficient, energy-saving, and environment-friendly is prerequisite for the development for biotechnology.

Supercritical fluid technology has received wide attention in recent years. It is known that a substance has 3 phases, namely, solid, liquid and gas. When the temperature and the pressure of a system reach a certain point, the density of liquid phase becomes the same as that of gas phase and a homogenous phase is thus formed. The point at which liquid phase and gas phase has the same density is called “critical point”, and its corresponding temperature, pressure and density are called critical temperature (Tc), critical pressure (Pc) and critical density (ρc), respectively. Once a substance reaches the critical point, no liquefaction or gasification will further occur even higher pressure or more heat are applied thereto. A fluid at a temperature and a pressure respectively higher than its critical temperature and pressure is called “supercritical fluid”.

Supercritical fluid has diffusion ability similar to gas and solvating ability similar to liquid. The solvating ability can be changed by changing the temperature, pressure and polarity of the supercritical fluid. Supercritical liquid also has low viscosity and low surface tension and can easily penetrate into materials with fine porosity. The aforesaid properties make supercritical fluid suitable for use in various fields such as non-aqueous cleaning, extraction, dyeing etc. For example, the supercritical carbon dioxide has been used to extract the caffeine and the herbal essence in German.

Carbon dioxide reaches the critical point easily. Its critical temperature is near the room temperature, i.e. about 31.1° C., and its critical pressure is about 72.9 bars. Furthermore, carbon dioxide is an odorless, colorless, nonflammable, nontoxic and nonpolluting gas. In addition, it is inexpensive, commercially and industrially available, and can be easily recycled and reused. Therefore, supercritical carbon dioxide is an excellent choice for a supercritical fluid.

Moreover, carbon dioxide is acidic when dissolved in water and has a bactericidal effect due to its acidic nature. Supercritical carbon dioxide has been applied in for sterilizing medical devices and the like as it could penetrate the cellular membrane of bacteria and exerted a bactericidal effect.

Nevertheless, the effectiveness of supercritical carbon dioxide or other supercritical fluids in inactivating viruses was unclear. Furthermore, supercritical fluids have never been used in sterilizing biomaterials having biological activities. It was not yet known whether the biological activities of biomaterials would decrease or lose after treatment with supercritical fluids.

### SUMMARY OF THE INVENTION

In light of the above prior-art problems, an object of the present invention is to provide a method for effectively inactivating viruses associated with or in biomaterials.

Another object of the present invention is to provide a method for inactivating viruses associated with or in biomaterials at lower temperature.

Still another object of the present invention is to provide a method for inactivating virus in a heat-unstable biomaterial such as a proteinous material, without decreasing the biological activities of said biomaterial significantly.

In order to achieve the foregoing and other objects, the present invention provides a method for inactivating viruses in biomaterials characterized in comprising a step of introducing a high-pressure fluid into a container containing a biomaterial at a speed capable of forming non-turbulent flow with Reynold number of 2000 or less, so as to inactivating viruses potentially existing in the biomaterial.

In a preferred embodiment, supercritical carbon dioxide is used as a high-pressure fluid.

In another preferred embodiment, supercritical carbon dioxide is used in an amount of 100 to 500 g relative to 1 g of the biomaterial to be treated.

In another preferred embodiment, supercritical carbon dioxide is introduced to the container at a pressure of about 60 to about 240 bars.

In another preferred embodiment, supercritical carbon dioxide is introduced to the container at a temperature of about 40 to about 80° C.

In another preferred embodiment, a co-solvent is added to the high-pressure fluid.

In another preferred embodiment, a microbial inhibitor is added to the high-pressure fluid.

### DETAILED DESCRIPTION OF THE PREFERRED EMBODIMENTS

Preferred embodiments of a method of inactivating viruses in biomaterials are described in detail hereinafter, and other technical features and advantages will be easily understood form the disclosure of the present invention by a person having ordinary skill in the art.

According to the present invention, provided is a method for inactivating viruses in biomaterials characterized in comprising a step of introducing a high-pressure fluid into a container containing a biomaterial at a speed capable of forming non-turbulent flow with Reynold number of 2000 or less, thereby inactivating viruses potentially existing in the biomaterial.

The term “high pressure fluid” used herein includes supercritical fluid, critical fluid, near critical fluid and liquefied fluid. The term “supercritical fluid” (SCF) used herein means a fluid with a temperature higher than a critical temperature and a pressure higher than a critical pressure. The term “critical fluid” used herein means a fluid with a temperature equal to critical temperature and a pressure equal to critical pressure. The term “near critical fluid” means a fluid with a temperature near critical temperature and a pressure near critical pressure.

Similarly, the term “high-pressure carbon dioxide” used herein includes supercritical carbon dioxide, critical carbon dioxide, near critical carbon dioxide and liquefied carbon dioxide.

The term “biomaterial” used herein includes, but not limited to, proteins, peptides, nucleic acids, bioactive molecules, platelets and blood factors and the like.

Reynold number (Re) is an indicator for a status of fluid dynamics. It is defined by the following formula (I):

$$Re = \frac{D \overline{u} ρ}{μ} \qquad ( I )$$

wherein,

D represents the interior diameter of the pipe expressed by meters;

\overline{μ}| represents average speed expressed by m/s;

ρ represents density expressed by kg/m³ ; and

μ represents viscosity expressed by kg/m·s.

A status of fluid with Reynold number smaller than 2,100 is called a laminar flow wherein the fluid layers are parallel to each other without turbulence when the fluid flows forwardly. A status of fluid with Reynold number larger than 4,000 is called a turbulent flow wherein the flow will become turbulent with the formation of eddies and chaotic motion. A status of fluid with Reynold number between 2,100 and 4,000 is called a transitional flow that is an intermediate state between a laminar flow and a turbulent flow, wherein the flowing behavior of fluid becomes unstable resulting a period of a predominated laminar flow or a period of a predominated turbulent flow occasionally.

According to the present invention, the high-pressure fluid is introduced at a speed that will not cause a turbulent flow. Therefore, it is obvious that inactivation of viruses is accomplished by a supercritical fluid rather than a turbulent flow in the present invention.

According to the present invention, the fluid suitable for use as the source of high-pressure fluid is selected from the group consisting of carbon dioxide, water, propane, xenon, nitrous oxide, hydrogen gas and chlorine gas. A high-pressure fluid is preferably a high-pressure carbon dioxide such as supercritical carbon dioxide or liquefied carbon dioxide, more preferably supercritical carbon dioxide.

According to the present invention, supercritical carbon dioxide is introduced to the container preferably in an amount of about 100 to 500 g, more preferably about 300 g, relative to 1 g of the untreated biomaterial.

According to the present invention, supercritical carbon dioxide is introduced to the container preferably at a pressure of about 60 to about 240 bars, more preferably about 150 to about 190 bars and most preferably about 160 bars.

According to the present invention, supercritical carbon dioxide is introduced to the container preferably at a temperature of about 40 to about 80° C., more preferably about 40 to about 60° C. and most preferably about 40 to about 50° C.

According to the present invention, the period of treating biomaterials depends on the temperature and the kind of the biomaterial to be treated, and it usually takes about two hours or less, preferably 1 hour or less when supercritical carbon dioxide is used as high-pressure fluid.

A co-solvent may be added, if necessary, to the high-pressure fluid to elevate the solvating ability of the fluid. The co-solvent is, for example, an organic solvent, such as acetone, hexane, dioxane, benzene, toluene, ethyl acetate, methanol, ethanol, acetonitrile, dimethylformamide, cyclohexane, trichloromethane, dichloromethane, pyridine, ethyl ether, nitromethane or anisole.

A microbial inhibitor may also be added, if necessary, to the high-pressure fluid. The microbial inhibitor is, for example, a peroxide such as hydrogen peroxide and peracetic acid, an aldehyde such as formaldehyde glutaraldehyde and orthphthaladehyde, a halogen-containing reagent such as iodine, Sterilox, ethanol, an acid, an alkali and the like. The amount and the timing of adding a co-solvent or a microbial inhibitor can be easily determined by a person of ordinary skill in the art.

The method of the present invention not only inactivates but also eliminates the viruses potentially existing in biomaterials. Furthermore, the method of the present invention can inactivate enzymes or biological active molecules associated with viruses and consequently suppress spreading and reproduction of viruses through inhibiting viral protease.

### EXPERIMENTS 1 TO 3 AND COMPARATIVE EXPERIMENT 1

Inactivation of Coronavirus

Materials:

ST cell line (ATCC CRL-1746)—114^{th} generation, and a Corona virus, i.e. Taiwanese wild strain (TFI) of transmissible gastroenteritis virus (TGEV) were used for test.

Method:

Preparation of samples: A TGEV solution with a titer of 10⁸ TCID₅₀/ml were thawed and added to an equal volume of 8% (w/v) gelatin, and then dispensed evenly into small glass bottles. After gelatin coagulated, the glass bottles were sealed and preserved at 4° C. until use.

Treating procedures: In Experiments 1 to 3, supercritical carbon dioxide, in an amount of 300 g relative to 1 g of the sample, was introduced into each bottle containing the sample at a speed capable of forming a non-turbulent flow with Reynold number of 2000 or less, under the condition listed in Table 1. In Comparative Experiment 1, the sample was not treated.

Determination of virus titer: After treatment, the samples were collected and centrifuged. The supernatant of each sample was taken and added to the wells of a 96-well microplates containing ST cell. The viral titer of each sample was determined and expressed by TCID₅₀, which was calculated by using Reed-Muench Method. Determination of virus titer was repeated for 6 times for each sample and their geometric mean (GM) is calculated by the following equation:
GM_{\overline{y}}=ⁿ\sqrt{y₁y₂y₃ . . . yₙ}

The results were listed in Table 1.

TABLE 1

| Experiment No. | Condition of treatment | Virus Titer** |
|---|---|---|
| Experiment 1 | 40° C., 160 bar, 30 mins | <10^{0.69}* |
| Experiment 2 | 50° C., 160 bar, 30 mins | <10^{0.69}* |
| Experiment 3 | 40° C., 160 bar, 60 mins | <10^{0.69}* |
| Comparative Experiment 1 | Untreated | 10^{6.68} |

*detection limit: 10^{0.69}/0.1 ml
**Virus titer is expressed by the geometric mean of TCID₅₀ values of six determinations.

### EXPERIMENTS 4 TO 6 AND COMPARATIVE EXPERIMENT 2

Inactivation of Porcine Reproductive and Respiratory Syndrome Virus

Material:

MARC-104 cell line and MD006 strain of porcine reproductive and respiratory syndrome virus (PRRSV) were used for this test.

Method:

Preparation of samples: A PRRSV solution with a titer of 10^{7.5}TCID₅₀/ml were thawed and then added to equal volume of 8% (w/v) gelatin, and dispensed evenly into small glass bottles. After gelatin coagulated, the glass bottles were sealed and preserved at 4° C. until use.

Treating procedures: In experiments 4 to 6, treatment was performed in the same manner as in experiments 1 to 3 except under the condition listed in Table 2. In comparative experiment 2, the sample was not treated.

Determination of virus titer: The virus titer of each sample was determined in the same manner as in Experiments 1 to 3, and the results are listed in Table 2.

TABLE 2

| Experiment No. | Condition of treatment | Virus Titer** |
|---|---|---|
| Experiment 4 | 40° C., 160 bar, 60 mins | <10¹* |
| Experiment 5 | 45° C., 160 bar, 60 mins | <10¹* |
| Experiment 6 | 50° C., 160 bar, 30 mins | <10¹* |
| Comparative Experiment 2 | Untreated | 10^{3.64} |

*detection limit: 10¹/0.1 ml
**Virus titer is expressed by the geometric mean of TCID₅₀ values of six determinations.

### EXPERIMENTS 7 TO 9 AND COMPARATIVE EXPERIMENT 3

Inactivation of Japanese encephalitis virus

Material:

Vero cell line and AT vaccine strain of Japanese encephalitis virus (JEV) were used for this test.

Method:

Preparation of samples: A JEV solution with a titer of 10^{7.1}TCID₅₀/ml were thawed and added to equal volume of 8 % (w/v) gelatin, and then dispensed evenly into small glass bottles. After gelatin coagulated, the glass bottles were sealed and preserved at 4° C. until use.

Treating procedures: In Experiments 7 to 9, treatment was performed in the same manner as in Experiments 1 to 3 except under the condition listed in Table 3. In Comparative Experiment 3, the sample was not treated.

Determination of virus titer: The virus titer of each sample was determined in the same manner as in Experiments 1 to 3, and the results are listed in Table 3.

TABLE 3

| Experiment No. | Condition of treatment | Virus Titer** |
|---|---|---|
| Experiment 7 | 40° C., 60 mins, 160 bar | <10¹* |
| Experiment 8 | 45° C., 60 mins, 160 bar | <10¹* |
| Experiment 9 | 50° C., 30 mins, 160 bar | <10¹* |
| Comparative Experiment 3 | Untreated | 10^{3.84} |

*detection limit: 10¹/0.1 ml
**Virus titer is expressed by the geometric mean of TCID₅₀ values of six determinations.

### EXPERIMENTS 10 TO 12 AND COMPARATIVE EXPERIMENTS 4 TO 5

Inactivation of Pseudorabies virus

Material:

RK cell line and Taiwanese wild strain of Pseudorabies virus (PRV) were used for test.

Method:

Preparation of samples: A PRV solution with a titer of 10^{6.3} TCID₅₀/ml were thawed and then added to equal volume of 8% (w/v) gelatin, and dispensed evenly into small glass bottles. After gelatin coagulated, the glass bottles were sealed and preserved at 4° C. until use.

Treating procedures: In Experiments 10 to 12 and Comparative Experiment 5, treatment was performed in the same manner as in Experiments 1 to 3 except under the condition listed in Table 4. In Comparative Experiment 4, the sample was not treated.

Determination of virus titer: The virus titer of each sample was determined in the same manner as in Experiments 1 to 3, and the results are listed in Table 4.

TABLE 4

| Experiment No. | Condition of treatment | Virus Titer** |
|---|---|---|
| Experiment 10 | 40° C., 160 bar, 60 mins | <10¹* |
| Experiment 11 | 45° C., 160 bar, 60 mins | <10¹* |
| Experiment 12 | 50° C., 160 bar, 30 mins | <10¹* |
| Comparative Experiment 4 | Untreated | 10^{4.1} |
| Comparative Experiment 5 | 45° C., 30 mins | 10^{3.5} |

*detection limit: 10¹/0.1 ml
**Virus titer is expressed by the geometric mean of TCID₅₀ values of six determinations.

### EXPERIMENTS 13 TO 15 AND COMPARATIVE EXPERIMENTS 6 TO 7

Effect of Supercritical Carbon Dioxide on Proteins having Biological Activities

Material:

MARC-104 cell line, MD006 strain of PRRSV, and hyperimmune antiserum against TGEV were used for this test.

Method:

Preparation of samples: 20 ml of PRRSV solution with a titer of 10^{7.5}TCID₅₀/ml were thawed and mixed with 20 ml of 8% (w/v) solution of gelatin in distilled water and 1 ml of TGEV antiserum to form a homogenous mixture. The mixture was dispensed evenly into small glass bottles. After gelatin coagulated, the glass bottles were sealed and preserved at 4° C. until use.

Treating procedures: In Experiments 13 to 15 and Comparative Experiment 7, treatment was performed in the same manner as in Experiments 1 to 3 except under the condition listed in Table 5. In Comparative Experiment 6, the sample was not treated.

Determination of virus titer: The virus titer of each sample was determined in the same manner as in Experiments 1 to 3.

Determination of TGEV neutralizing antibody titer: TGEV neutralizing antibody titer of each sample before and after treatment was determined according to OIE rules. A 100 μl was taken from each sample and 2-fold serial dilution (from 2⁻¹ to 2⁻¹²) was made. Then a 100 μl of 100 TCID₅₀ TGEV was added to each diluted samples and incubated for an hour. After incubation, the mixture was transferred to wells of a 96-well microplates containing the cell lines grown to confluence and incubated for five days. Then, neutralizing antibody titer was determined.

Determination of virus titer and determination of neutralizing antibody titer were repeated respectively for 5 times in Experiments 13 to 15, 2 times in Comparative Experiment 6, and 3 times in Comparative Experiment 7. The results are listed in Table 5.

TABLE 5

| | | Viral Titer | | Neutralizing | |
|---|---|---|---|---|---|
| | | (TCID₅₀) | | Antibody Titer | |
| | | Value of | | Value of | |
| Experiment | Conditions of | Individual | | Individual | |
| No. | Treatment | Determination | GM** | Determination | GM** |
| Experiment 13 | 40° C., | <10¹* | <10¹ | 362 | 588 |
| | 160 bar, | <10¹* | | 512 | |
| | 60 mins | <10¹* | | 512 | |
| | | <10¹* | | 724 | |
| | | <10¹* | | 1024 | |
| Experiment 14 | 45° C., | <10¹* | <10¹ | 724 | 675 |
| | 160 bar, | <10¹* | | 724 | |
| | 45 mins | <10¹* | | 1024 | |
| | | <10¹* | | 724 | |
| | | <10¹* | | 362 | |
| Experiment 15 | 50° C., | <10¹* | <10¹ | 362 | 548 |
| | 160 bar, | <10¹* | | 512 | |
| | 30 mins | <10¹* | | 724 | |
| | | <10¹* | | 724 | |
| | | <10¹* | | 512 | |
| Comparative | Untreated | 10^{3.5} | 10^{3.5} | 724 | 724 |
| Experiment 6 | | 10^{3.5} | | 724 | |
| Comparative | 45° C., | 10^{3.32} | ¹⁰^{3.32} | 362 | 574 |
| Experiment 7 | 30 mins | 10^{3.5} | | 512 | |
| | | 10^{3.15} | | 1024 | |

*detection limit: 10¹/0.1 ml
**GM: geometric mean

### EXPERIMENTS 16 TO 18 AND COMPARATIVE EXPERIMENTS 8 TO 9

Effect of Supercritical Carbon Dioxide on Proteins having Biological Activities

Material:

RK cell line, Taiwanese wild strain of PRV, and hyperimmune antiserum against TGEV were used for test.

Method:

Preparation of samples: In Experiments 16 to 18, 20 ml of PRV solution with a titer of 10⁸TCID₅₀/ml were thawed and mixed with 20 ml of 8% (w/v) solution of gelatin in distilled water and 1 ml of TGEV antiserum to form a homogenous mixture. The mixture was dispensed evenly into small glass bottles. After gelatin coagulated, the glass bottles were sealed and preserved at 4° C. until use. In the samples of Comparative Experiments 8 and 9, PRV was not added.

Treating procedures: In Experiments 16 to 18 and Comparative Experiment 9, treatment was performed in the same manner as in Experiments 1 to 3 except under the condition listed in Table 6. In Comparative Experiment 8, the sample was not treated.

Determination of virus titer: The virus titer of each sample was determined in the same manner as in Experiments 1 to 3.

Determination of neutralizing antibody titer: The neutralizing antibody titer of each sample was determined in the same manner as in Experiments 13 to 15.

Determination of virus titer and determination of neutralizing antibody titer were repeated respectively for 5 times in Experiments 16 to 18, 2 times in Comparative Experiment 8, and 3 times in Comparative Experiment 9. The results are listed in Table 6.

TABLE 6

| | | Viral Titer | | Neutralizing | |
|---|---|---|---|---|---|
| | | (TCID₅₀) | | Antibody Titer | |
| | | Value of | | Value of | |
| Experiment | Conditions of | Individual | | Individual | |
| No. | Treatment | Determination | GM** | Determination | GM** |
| Experiment 16 | 40° C., | <10¹* | <10¹ | 362 | 445 |
| | 160 bar, | <10¹* | | 362 | |
| | 60 mins | <10¹* | | 362 | |
| | | <10¹* | | 512 | |
| | | <10¹* | | 724 | |
| Experiment 17 | 45° C., | <10¹* | <10¹ | 724 | 512 |
| | 160 bar, | <10¹* | | 362 | |
| | 45 mins | <10¹* | | 512 | |
| | | <10¹* | | 724 | |
| | | <10¹* | | 362 | |
| Experiment 18 | 50° C., | <10¹* | <10¹ | 256 | 362 |
| | 160 bar, | <10¹* | | 362 | |
| | 30 mins | <10¹* | | 512 | |
| | | <10¹* | | 362 | |
| | | <10¹* | | 362 | |
| Comparative | No PRV is added; | — | | 724 | 724 |
| Experiment 7 | Untreated | — | | 724 | |
| Comparative | No PRV is added; | — | — | 362 | 574 |
| Experiment 8 | 45° C., 30 mins | — | — | 512 | |
| | | — | — | 1024 | |

*detection limit: 10¹/0.1 ml
**GM: geometric mean

It can be seen from the Tables 1 to 6 that the virus titers of all samples of Experiments 1 to 16 according to the present invention decrease to a undetectable level after treatment with supercritical carbon dioxide under the pressure of 160 bar and the temperature of 40 to 50° C. for 30 to 60 min. However, the virus titers of the samples of Comparative Experiments 5 and 7, after treatment with carbon dioxide at normal pressure and 45° C. for 30 min, do not decrease or merely slightly decrease compared to those of untreated samples of Comparative Experiments 4 and 6.

Furthermore, as shown in Tables 5 and 6, treatment with supercritical carbon dioxide under the conditions used does not cause a significant decrease in the neutralizing antibody titer of co-existing TGEV antiserum.

Therefore, it can be concluded from the above results that supercritical carbon dioxide can effectively inactivate viruses in a heat-sensitive biomaterial such as proteinous material without significantly decreasing the biological activities of said biomaterial.

The invention has been described using exemplary preferred embodiments. However, it is to be understood that the scope of the invention is not limited to these embodiments. Any modification and variation can be made without departing the spirit of the present invention, and all such modification and variation are included in the scope of claims.

### Value of Industrial Utilization

According to the present invention, a safe, effective, non-polluting and economic method of inactivating viruses potentially existing in biomaterials without significantly decreasing the biological activities of the biomaterials after treatment is provided.

What is claimed:

1. A method of inactivating viruses associated with a biomaterial, characterized in comprising a step of introducing a high-pressure fluid into a container containing a biomaterial to be treated at a speed capable of forming a non-turbulent flow with Reynold number of 2000 or less, so as to inactivating viruses potentially existing with the biomaterial.

2. The method of claim 1, wherein the fluid used as the source of a high-pressure fluid is selected from the group consisting of carbon dioxide, water, propane, xenon, nitrous oxide, hydrogen gas and chlorine gas.

3. The method of claim 2, wherein the high-pressure fluid is supercritical carbon dioxide.

4. The method of claim 3, wherein the high-pressure carbon dioxide is liquefied carbon dioxide.

5. The method of claim 3, wherein supercritical carbon dioxide is introduced to a container in an amount of 100 to 500 g relative to 1 g of the untreated biomaterial.

6. The method of claim 5, wherein supercritical carbon dioxide is introduced to the container in an amount of 300 g relative to 1 g of the untreated biomaterial.

7. The method of claim 3, wherein supercritical carbon dioxide is introduced to the container at a pressure of about 60 to about 240 bar.

8. The method of claim 3, wherein supercritical carbon dioxide is introduced to the container at a pressure of about 100 to about 200 bar.

9. The method of claim 3, wherein supercritical carbon dioxide is introduced to the container at a pressure of about 150 to about 190 bar.

10. The method of claim 3, wherein supercritical carbon dioxide is introduced to the container at a pressure is about 160 bars.

11. The method of claim 3, wherein supercritical carbon dioxide is introduced to the container at a temperature of about 40 to about 80° C.

12. The method of claim 11, wherein supercritical carbon dioxide is introduced to the container at a temperature of about 40 to about 60° C.

13. The method of claim 12, wherein supercritical carbon dioxide is introduced to the container at a temperature of about 40 to about 50° C.

14. The method of claim 1, wherein the high-pressure fluid further contains a co-solvent.

15. The method of claim 14, wherein the co-solvent is an organic solvent.

16. The method of claim 15, wherein the co-solvent is at least one selected from the group consisting of acetone, hexane, dioxane, benzene, toluene, ethyl acetate, methanol, ethanol, acetonitrile, dimethylformamide, cyclohexane, trichloromethane, dichloromethane, pyridine, ethyl ether, nitromethane and anisole.

17. The method of claim 1, wherein the high pressure fluid further contains a microbial inhibitor.

18. The method of claim 17, wherein the microbial inhibitor is at least one selected from the group consisting of peracetic acid, hydrogen peroxide, glutaraldehyde, ortho-phthaladehyde, iodine and ethanol.

19. The method of claim 1, wherein the virus includes Coronavirus, Porcine reproductive and respiratory and respiratory syndrome virus, Japanese encephalitis virus and Pseudorabies virus.

20. The method of claim 1, wherein the biomaterial is a material having biological activities.

21. The method of claim 20, wherein the material having biological activities is protein, peptide, nucleic acid, bioactive molecule, platelet or blood factor.
